(19) United States
(12) Patent Application Publication
(10) Pub. No.: US 2021/0065296 A1
(43) Pub. Date: Mar. 4, 2021

(54) INTELLIGENT TRADING AND RISK MANAGEMENT FRAMEWORK

(71) Applicant: Ziggurat Technologies, Inc., Berkeley, CA (US)

(72) Inventors: Siamak NAZARI, Berkeley, CA (US); Hassan KHANIANI, Berkeley, CA (US)

(21) Appl. No.: 17/096,969

(22) Filed: Nov. 13, 2020

Related U.S. Application Data
(63) Continuation-in-part of application No. 16/365,631, filed on Mar. 26, 2019.
(60) Provisional application No. 62/934,535, filed on Nov. 13, 2019.
(60) Provisional application No. 62/648,154, filed on Mar. 26, 2018.
(60) Provisional application No. 62/701,851, filed on Jul. 23, 2018.

(51) Int. Cl. G06Q 40/04; G16H 40/67; G06Q 40/08; G06N 20/00

(52) U.S. Cl. CPC G06Q 40/04; G16H 40/67; G06Q 40/02; G06N 20/00; G06Q 40/08

(57) ABSTRACT

A computer-implemented integrated framework for managing real-time financial trades and risk management is described herein. Quantitative and sentimental parameters of trading market are identified and analyzed. A stock selection module having a deep learning architecture performs future predictions based on the analyzed quantitative and sentimental parameters, wherein the stock selection module comprises. A probability number in percentage is assigned to a trading decision. Based on the assigned probabilities to different trading decisions, entrance and exit signals are provided to a user based on their preferences such as the user's risk tolerance.

### CROSS-REFERENCE TO RELATED APPLICATIONS

This application claims the benefit of co-pending U.S. Provisional Application Ser. No. 62/934,535, filed on Nov. 13, 2019, and is also a continuation-in-part application which claims benefit of co-pending U.S. patent application Ser. No. 16/365,631, filed on Mar. 26, 2019, which claims the benefit of U.S. Provisional Application Ser. No. 62/648,154, filed on Mar. 26, 2018, and claims the benefit of U.S. Provisional Application Ser. No. 62/701,851, filed on Jul. 23, 2018, which are herein incorporated by reference in their entireties for all purposes.

### FIELD OF THE INVENTION

The present disclosure relates to an investment or intelligent trading risk management platform which takes into account the risk profile of an investor and overall market sentiment.

### BACKGROUND

Investing in financial instruments, such as equities, commodities, options, bonds, as well as others involves a significant amount of risks. To reduce financial risks, financial market data is available to assist an investor in deciding which financial instruments to invest. The current available financial data is enormous, both in terms of quantity and type due to the internet. In addition, access to different markets is unprecedented. For an average investor, it is very difficult to sift through the enormity of data and determine which financial instruments or assets to invest as well as identifying arbitrage opportunities.

In addition, different investors have different risk profiles. For example, some investors are high-risk takers while others are low-risk takers. Also, investors may have different sentiments (psychology) for the buy-side and sell-side (overall market sentiment).

Quantitative models have been employed by, for example, trading platforms to reduce risks or identify investment opportunities. However, current quantitative models used by trading platforms fail to take risk profiles of investors as well as market sentiments into consideration when recommending instruments.

From the foregoing discussion, it is desirable to provide an intelligent trading risk management platform that takes into account overall market sentiments as well as the risk profile of an investor.

### SUMMARY

Embodiments of the present disclosure generally relate to a trading risk management platform. In particular, the present disclosure relates to an intelligent trading risk management platform which takes into account the risk profile of the investor and overall market sentiment.

In one embodiment, a method for trading and risk management includes identifying quantitative and sentimental parameters of trading market, receiving bio-signals including natural responses of a user, analyzing the quantitative and sentimental parameters of the trading market, providing a stock selection module to perform future predictions based on the analyzed quantitative and sentimental parameters and the stock selection module comprises a deep learning architecture. The method further includes providing a probability number in percentage to a trading decision, providing entrance and exit signals to the user based on the user's preferences, and wherein the received bio-signals are configured to verify the trading decision and customize the analyzing based on the user's preferences.

These and other advantages and features of the embodiments herein disclosed, will become apparent through reference to the following description and the accompanying drawings. Furthermore, it is to be understood that the features of the various embodiments described herein are not mutually exclusive and can exist in various combinations and permutations.

### BRIEF DESCRIPTION OF THE DRAWINGS

The accompanying drawings, which are incorporated in and form part of the specification in which like numerals designate like parts, illustrate preferred embodiments of the present disclosure and, together with the description, serve to explain the principles of various embodiments of the present disclosure.

FIG. 1 shows a simplified diagram of a backend of an intelligent trading risk management platform;

FIG. 2 shows an exemplary embodiment of a process performed by the intelligent trading risk management platform;

FIG. 3 shows a panel with user preferences;

FIG. 4 shows an exemplary architecture of a stock selection module;

FIG. 5 shows an exemplary matrix of investment opportunities for stock selection based on the analysis performed by the platform;

FIGS. 6a and 6b show examples of Sentiment analysis using different signals;

FIG. 7 shows an exemplary embodiment an intelligent trading risk management process;

FIG. 8 shows an embodiment of a deep learning architecture for stock prediction;

FIG. 9 show different examples of future predictions for different time frames;

FIG. 10 shows an embodiment of a device for recording information of the user; and

FIG. 11 shows a correlation of heart rate, blood pressure, EEG and price chart in time.

### DETAILED DESCRIPTION

Embodiments generally relate to a trading risk management trading platform or framework. In particular, the trading platform is an intelligent platform that makes recommendations based on a personal risk profile as well as overall market sentiment. The intelligent platform employs advanced mathematical modeling, artificial intelligence, machine learning, and deep learning, as well as other existing trading instruments as a leverage tool to quantify uncertainty and manage the risk in the prediction of the asset price and trends of different asset classes and charts.

The platform reduces the uncertainty of investment or trading decision making by measuring the sentiment (e.g. psychology) of the markets as well as the behavior of buyers and sellers and translates them into probabilities of edge versus odds. The platform also verifies the way the predictive model is showing the direction of the price action as the order size on bid (e.g. buy) and ask (e.g. sell) price and volume change. Level II (e.g. tick data or the price and size of buying and selling orders) market data may help to understand the behavior of the price since the force behind the price movement is psychological changes of buyers and sellers.

The platform minimizes the uncertainty of trade decisions and quantifies the risk in the form of probability within a range of 1-99% win or loss by providing entry and exit signals in and out of the market. For example, the platform manages market-timing risk.

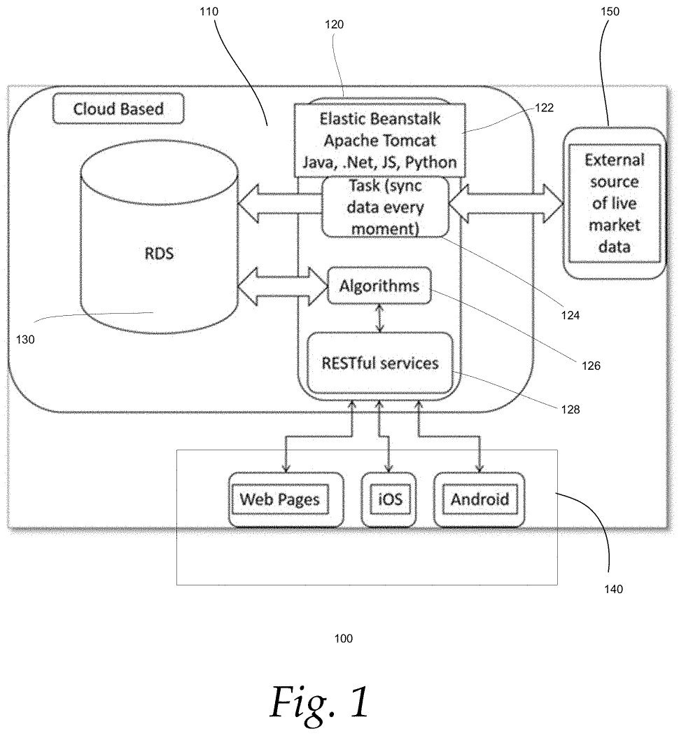

FIG. 1 shows a simplified diagram of an intelligent trading risk management platform 100. The platform, for example, may be a software application (App) having a frontend 140 and a backend 110. A user may access the App by registering an account. User information, including login information as well as the profile information may be maintained by the backend.

The frontend of the platform runs on a client device. For example, clients access the backend of the platform through client devices. The client device, for example, may be a client computing device. The client device may be a mobile or non-mobile computing device, such as a laptop, a tablet a smartphone, or a desktop computer. For example, the client devices may run on IOS, Android, or PC operating systems. Other types of computing devices may also be useful. In one embodiment, the App is web-based. Other types of Apps, such as native or hybrid Apps, may also be useful. Other types of Apps may also be useful.

As for the backend 110, it runs on a backend server. For example, a backend of the platform may reside on a server. A server may include one or more server computers. As shown, the trading risk management platform is a cloud-based platform. For example, the backend resides on a server in the cloud.

In one embodiment, the backend includes a storage module 130 and a processing module 120. The storage module, for example, may be a relational database service (RDS). Other types of storage modules may also be useful. As for the processing module, it includes a runtime environment unit 122, a task unit 124, an analytics unit 126, and an interface unit 128.

In one embodiment, the runtime environment unit is configured as a web application server running web programs. For example, the web application server may include Apache Tomcat which runs Java servlets as well as other programs, such as .Net, Javascript, and Python programs. The runtime environment may also include web application services, such as Elastic Beanstalk. Other types of web application services may also be useful.

The task unit is configured to receive data from external data sources 150. The data, for example, includes financial data, such as market data and data from exchanges. Other data, such as news, politics, social media, and other data that may impact the markets may also be useful. Data can be obtained from sources through subscription services, data scraping and data crawling, as well as other data acquisition techniques. The task unit processes the external data and stores it in the storage module. Processing, for example, may include categorizing the data, date stamping, and tagging. Other types of processing may also be performed.

The interface unit facilities communication between client devices and the backend. For example, the interface unit may include web services for communication between the client devices and the backend. The interface unit may include RESTful services. Other types of web services may also be useful.

The analytics unit performs analytics on the processed data in the storage module. Analytics may employ advanced mathematical models, artificial intelligence, machine learning, deep learning, and various types of neural network models, such as CNNs and RNNs. The analytics unit processes the data to perform future value predictions. The result of the analytics is stored in the storage module.

In addition, the analytics module may provide the results to the user. For example, analytics may be performed on data based on a user request and the results are presented to the client device of the user. For example, the end-user may select a real-time assets trading event. Real-time quantifiable assets assessment data feed is delivered from the system to the client device. The risk associated with each of these events is ranked and a probability number in percentage is assigned to each decision making on each trade.

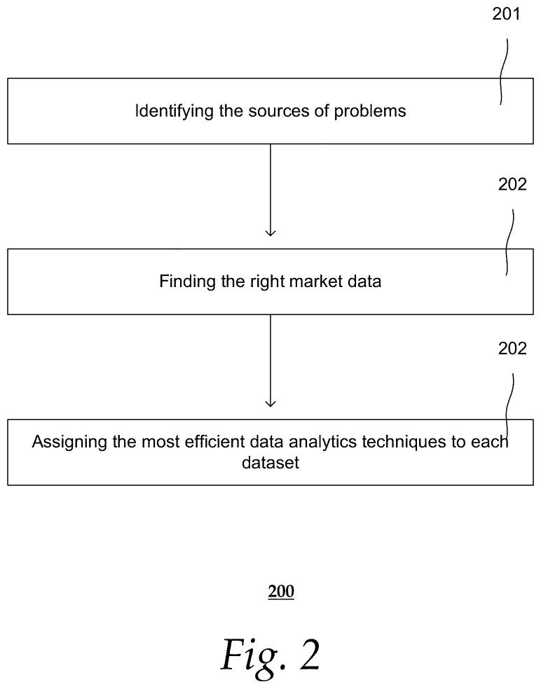

FIG. 2 shows an exemplary process performed by the intelligent trading risk management platform. The process, for example, identifies the sources of problems, finding the right market data, and assigning the most efficient data analytics techniques to each dataset. The process, for example, may be employed to assess financial risks.

The sources of risks every trader and investor is dealing with are defined quantitatively at 202. The sources of risks are then modeled.

The real-time and intelligent risk source identification, assessment, and management may be imposed by different events on the price of an asset. These events can be news, financial and economic reports, companies' earnings, major and minor natural and man-made disasters and events, sentiment and psychology change of the market, firms, and big investors in a quantifiable application. The risk associated with each of these events is ranked and a probability number in percentage is assigned to each decision making process on each trade.

The right datasets serving a specific problem are found at 202. Cross-correlation of a chart or an asset compared to the other assets may be found. The dependency of an asset price with respect to any other assets or stocks, markets, indices prices, and or market sentiments may also be identified. This ranks all the important factors affecting price of a single trade, an asset, or a portfolio in real-time, based on a weighting system. This weighting and factors list can change on daily basis.

At 203, the right analytics such as mathematical models, machine learning techniques, deep learning, and various types of neural network models, such as CNNs and RNNs as well as trading and portfolio management techniques are applied to each dataset to solve the market and investment risk problem. Highly accurate entry and exit, and or buy, hold or sell signals may be provided through a combination of quantitative and sentimental analytical approaches to constrain the risk and uncertainty in decision making associated with entering a trade or market, and exit out of it. For example, a multi-dimensional approach is applied and incorporates a few parameters into the calculation and consideration, such as the memory of the price action, stability of a real-time analysis, fast computational speed, cloud computational analysis, big data and deep learning algorithms, to find the patterns and anomalous behaviors in both price, volume and sentiment of the market or assets.

The platform implements several algorithms to quantify the uncertainty in the predictions of future assets values, and provides to the user important hints and advice to optimize entries and exits in a semi-automatic mode. The platform also has the capability of performing automatic trading once the personal risk profile of the trader has been defined.

As an example, the platform provides real-time analytics. For example, optimum entrance and exit signals are provided to the user. Optimal investment decisions are achieved by maximizing the number of right decisions and minimizing the number of wrong decisions. Maximizing the number of right decisions and minimizing the number of wrong decisions may be based on the amount of loss of capital induced by a wrong (precipitated) entrance.

The platform includes various modules. In one embodiment, the platform includes an AI stock picking and search engine. The stock selection is based on simulation of investing strategies and optimization of the maximum return and minimum risks of investments. In the AI algorithm, estimation of market potentials for investment and stimulation of the expected reward and the associated risk tolerance for each stock may be performed. A screener or search module is also designed to filter out different assets based on different search or filter criteria. These criteria can be either fundamental, technical, or quantitative factors. Especially when it comes to risk measurement, assets can be screened and selected based on their daily, short-term, and long-term risk of trading, as well as for buy and sell purposes. Moreover, this module would help to sort best LONG, SHORT and SIDEAWAY movement opportunities and based them on the risk of loss or reward of gain in real-time. The stocks that do not fit with the analytics, for example, those that give less or negative revenue, are eliminated from recommendations.

To manage the risk of market investment, specific AI computed zones (Entry zone, profit target zone and stop-loss zone) are generated. For example, these zones are defined by ranges for prices in which execution occurs. This real-time execution of orders ensures that the orders go through. As such, this avoids any limit-order which usually happens when banks and exchanges see those data and sell them to bigger players to crush small traders.

Seamless execution may also be performed through a list of brokers of choice. For example, the platform is paired and connected to existing brokerage firms or banks for direct sending of trade orders. Users can then focus on making the right move instead of switching between platforms and pages while trading.

Predictive analytics and Future Prediction through AI provides the prediction of what is going to happen, during a BUY, HOLD, or SELL decision, and gives the user sufficient time ahead to decide on the possible types of action and also their predictability index of it. The predictability index may refer to the plausibility of an opportunity as a hold or sell position, and can be quantified as a percentage.

A toolbox may be employed to provide a series of analytics to study, research, chart, and domestic and global market intelligence, and deep knowledge through machine intelligence, and human insights, and inputs.

Position Sizing feature provides an intelligent way to define how much capital and how many shares need to be bought or sold as an optimal solution. This is also another step towards managing the capital at risk per trade, and per account or portfolio. There are scenarios when a user aims to buy or sell a few tickers, or securities at the same time or during the same trading sessions. This module would help the user to allocate the entire capital sitting in his account intelligently and automatically. A few inputs or questions will be asked from the user to calculate this. Over time, the platform learns from the way the users assign the optimal share numbers to their account or trades, such as by depending on their state of behavior as well as the market conditions.

The platform may utilize trading journals to determine emotion and bias measurements. For example, a user may be asked to input whatever they know or heard or read about on the different stocks or assets into the platform. The input may also include their emotions, before, during, and after the trades, and the biases or sources of information they looked at when making the decisions to buy or sell.

The platform may include a Back-testing and Forward Simulation (FW-Sim) module. The module enables a user to choose indicators, build strategies, and apply it to the data in two different modes: (A) static data for back-testing (B) dynamic data for forward simulation. When a Back-testing process is done, a user receives a performance report, including (but not limited to) equity curve performance, Sharpe ratio, Risk Adjusted Return, Sortino Ratio, Gain to Loss ratio, Number of Win to Lose Trades over certain period of time and chosen time-period, etc. Forward Simulation may be performed to simulate the real-time analysis scenarios and calculate the same parameters mentioned above on the same time period. Eventually this report can be also exported, downloaded or saved in a format of choice (i.e. *.pdf, *.csv, *.txt or *.doc). This information can be saved inside the platform or as a file to be applied and tested to any chart or asset of choice. There may also be an option which can compare the static (back-testing mode) versus dynamic (forward simulation) data in terms of performance. This comparing option shows how good the strategy performs overall in the lifetime of the portfolio.

A Real-time-Analysis module includes charting tools, drawing, as well as quantitative-based indicators, and tools. Real-time data and its analysis is displayed on a panel. Windows may be added or removed from the panel. For example, added windows include windows with different boxes to type in different assets or for linking windows of choice to one another.

A Monitoring System in Realtime and a Alerting module may form part of the risk management framework to enable a trade or position to exit safely either with profit and or with minimum loss. This monitoring system would also provide insights and knowledge of existing great opportunities that can be swapped with existing ones in the user's portfolios, especially in the case of deciding how to execute the best practice

The platform may include an offline Data Analytics and Display module. For example, management of the module is displayed as a window. The displayed window may be similar to that of the real-time module. The difference is that advanced analytics, indicator and quantitative tools, and strategies can be developed here and applied either to back-testing/forward simulation and or real-time analysis panels.

A Risk Management Strategies module facilities and manages a user's Capital allocation risk. For example, “position sizing” is adapted to a user's style of trading, risk tolerance, behavior, etc. This module includes:

i) server-side algorithm
Filter & Sort
Selection

ii) realtime algorithm

iii) Client-side (selection) algorithm

Real-time position tracker module employs parameter settings, such as user risk tolerance, trading holding period such as day trading, swing trading, short-term trading, and or longer-term investing to defined quantitatively based on holding time duration. An alerting system is designed and parameters are set in this panel. Users can set risk tolerance numbers based on dollar amount or percentage (%). In the case when a dropdown happens, and or price upward and downward actions hit the risk thresholds, the alert is triggered and the user can receive a notification. The notification can be in the form of a text message, or an email altering text. This option can be turned on and off.

In one embodiment, the platform is configured to perform market analysis. For example, Sentiment Analysis and market trending are provided and shown in the panel. User can set all the parameters and turn on the sentiment analysis for it to be effective in the risk management solution.

Portfolio Management and Optimization module is a real-time realized and unrealized gains and loss on portfolio over certain period of time chosen by the user. It has the analytics that showing what, when, and where a trade went wrong, and what could have been done in terms of avoiding loss, or improving the strategy, etc. It is a diagnosis tool for every single trade, portfolio and or asset. There are some portfolio management optimization tools, taking advantage of different assets and strategies to minimize loss and or risk, and maximize returns using quant-based multi assets, and cross-assets allocation techniques.

An account management module maintains users' personal and account information, including profile information, names, emails, payment system, user, password changes, etc. are available and can be updated at any time. Included also are links to experts, such as tax and legal experts, which a user may desire access.

In one embodiment, the platform is cloud-based. This makes scalability, and state-of-the-art capacities of big data analysis easier, faster, and rapidly scalable to the global scale, using all 52+ active exchanges around the world. Cryptocurrencies as a global asset and their decentralized exchanges would also impose the risk of price quotes mismatch of the same cryptocurrency but in different exchanges. This risk along with market timing risk of trading these assets are also incorporated into the platform in order to provide real-time quantitative risk assessment solution and analytics.

The platform may be an Artificial intelligence kit. Several market parameters are optimized to maximize the returns and minimize the risks using training and testing procedures. The market data is considered as complex information that is not always predictable. It has both low and high frequency content which their patterns are important for prediction of the market behavior. The low frequency content is used for markets that are not volatile compared to the high frequency content that deals with higher volatility. The AI algorithm solves the complexity of market data comprising three steps, namely; forward simulation and filtering. The forward simulation simulates the trading over the past with the given analytics and initial parameters for zones of entry, profit, stop-loss and market memory. The market memory is a period of time where the implemented analytics has the best accuracy. For each parameter, the trading returns are calculated.

The platform employs pattern recognition for market investment with two major strategies of Single and multiple Market-Driven Algorithms.

A Single Market-Driven Algorithm is a market pattern prediction tool. This feature is based on the history of individual markets. We find an optimized market memory, where its similar pattern is searched throughout the history of market. The consequences of similar patterns in the market history is statistically analyzed for BUY/SELL/HOLD positions and are reported to the users in their decision making.

The framework calculates the several market indicators that depend on the market Open, Close, High and Low and Volume (e.gs., Average True Range (ATR), Accumulation/Distribution Line) of current and previous days. The super indicator is the best combination of all or parts of the indicators which is found such that they have the maximum probability to predict the market behavior and to have maximum return and minimum risks for investment. This feature is designed to automatically test the indicators but users may also be provided with with an option to select their desired indicators.

As for the Multiple Markets-Driven Algorithm, it includes downloading and analyzing thousands of stock worldwide every day. The algorithms study the market variation and cross plot the patterns of their parameters in decision making. The platform is able to find cross-correlation of one chart or asset compared to the other assets and identifies that a price of one asset for example a stock price is dependent on what other assets or stocks, markets, indices prices, and or markets sentiments. It ranks all the important factors affecting price of a single trade or asset, a portfolio, and asset in real-time, based on a weighting system. This weighting and factors list can change on daily basis. The advantage of this is that the behavior of some stocks in the market becomes predictable especially when the behavior of some of the stocks has a reverse correlation with each other.

The platform is an integrated and intelligent software solution, SaaS, for managing financial trades, portfolios, and asset risks in real-time, and in a more robust, quantitative and informative fashion. Different sources of data such as supply and demand function and behaviors, as well as news, reports, events would affect the behavior of every single asset in different markets. These factors create volatility in the market and create the uncertainty at any given moment in time. These different sources of data and behavior, impose uncertainty investment decision-making processes. The platform aims to solve for those uncertain sources of information which impose risk on every single trade, portfolio and or asset executed by traders, investors, and portfolio and asset managers, domestically within the US and or globally. It also helps to manage risks for globally allocated portfolios and assets through its comprehensive easy-to-use quant platform. The platform is able to identify, assess, and manage risk of a single trade to big scale assets, intelligently and in real-time, provide analytics which help investment decision-makers to minimize the risk of entering and exiting a trade, market, and or an asset.

The intelligent trading and risk management platform is able to provide accurate signaling for entry and exit, and for buying and selling of low volatility to very high volatility assets such as Cryptocurrencies. Intelligent Analytics not only provides entry and exit points but also provides a zone in order to buy or sell assets. This approach would help in mitigating of execution as well as timing risk. User may or may not miss an opportunity in order to make a decision at a point, but in this zone user still has time to make a decision to buy or sell without the feeling of missing out. This feeling usually makes the investor or trader feel missing out or has to chase the price action. Chasing is usually one of the main factors and reasons for making wrong decisions and mistakes.

Price and volume zoning capabilities would bypass the limit order functionaries offered by brokerage firms. This would help traders to be able to execute in realtime automatically without the hassle of getting their limit orders to be reported to the clearing houses and or exchanges. This capability would help to get the buy and or sell orders filled with realtime price and bid/ask feeds instead of setting at a certain level statistically and manually they it might never be executed. This would also eliminate the execution risk and user can always make sure order will go through as long as the range of price and spread is wide enough to be matched with bid or ask price and volume. ENTRY zone is for a range of price ad or volume where the trade opens, and PROFIT TARGET and STOP LOSS zones are for the range of prices and or volume that a trade or a group of trades close. These zones are dynamically set to user's best interests and desired risk tolerance and expected reward numbers.

The platform parameterizes user information to perform analysis in real-time, providing to the user optimum entrance and exit signals, and achieving optimal investment decisions by maximizing the number of right decisions and minimizing the number of wrong decisions, that is the amount loss of capital induced by a wrong (precipitated) entrance.

The platform picks stocks based on Machine Learning algorithms from risk %, Reward %, Risk-to-Reward ratio, probability of predictability, are developed. We call this next generation of intelligence level, to learn from A.I. itself.

The platform uses predictive trend analysis and optimizing the risk and return in order to confirm exits in the form of profit target zones and stop loss zones.

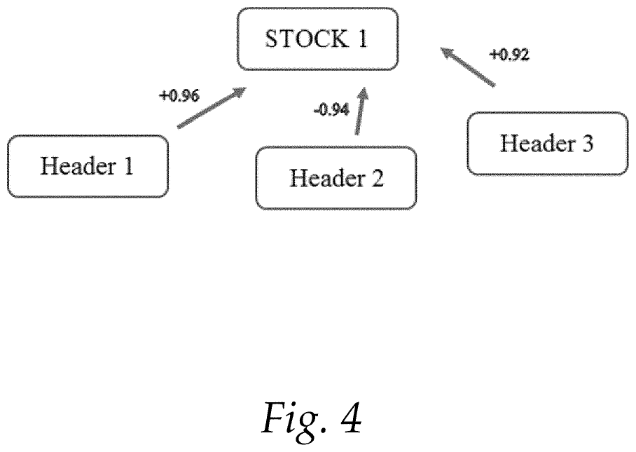

The platform finds, analyzes, executes, and monitors and finally exits one or multiple trades in real-time to establish a dynamic intelligent portfolio optimization for both short-term in the form of trades, and long term in the form of buy and hold positions. FIG. 4 shows such a framework.

The framework is capable of real-time and intelligent risk source identification, assessment, and management, imposed by different events on the price of an asset. These events can be news, financial and economic reports, companies' earnings, major and minor natural and man-made disasters and events, sentiment and psychology change of the market, firms, and big investors in a quantifiable fashion. Risk associated with each of these events is ranked and a probability number in percentage is assigned to each decision making on each single trade.

The platform's stock picking is based on real-time data mining and analysis procedure. The methodology identifies the optimum timeframe to monitor the stock and to generate the entry and exit zones as signals dynamically with notification systems as real-time position and portfolio tracker in place. This approach helps to drop and add those trade opportunities which would optimize the portfolio or list of holding positions and assets dynamically with and or with human approval or intervention.

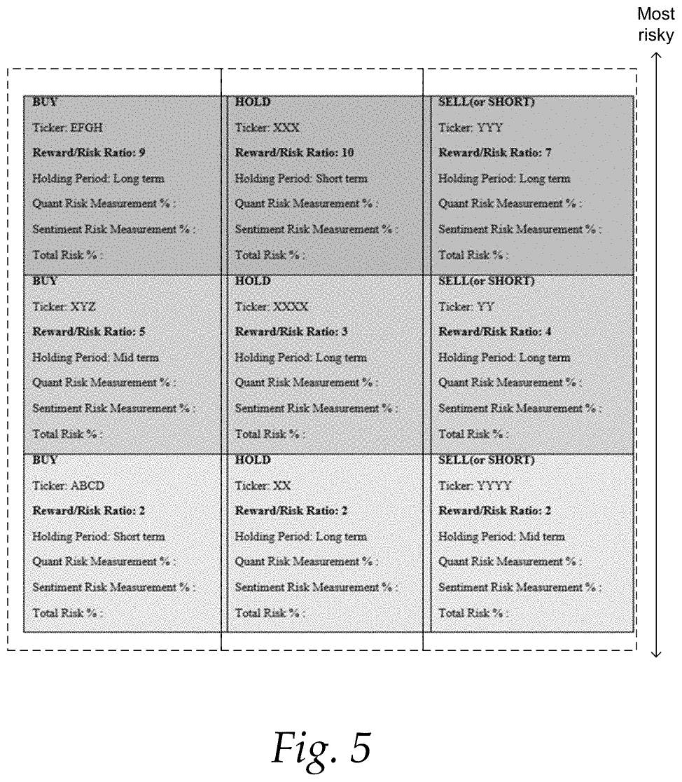

Use of machine learning techniques, deep learning, and various forms of neural network models including CNNS, RNNs, etc, combined with optimization techniques to design a neural net to create an intelligent architecture and perform future values of predictions. FIG. 5 shows such a technique where the middle layers are hidden variables need to be calculated based on known layers.

The platform provides future price predictions, including s in different time frames (minutes, days or weeks). The platform performs future predictions for various quantitative, and qualitative, fundamental, technical and sentiment parameters time in different time frames (minutes, days or weeks) with an associated probability of occurrence

The platform provides customizability of the zoning functionality. These zones can be adjusted based on best fit for risk to reward ratio which matches users trading and or investing style or inputs. AI algorithms learns from users style of investing, behaviors and other inputs to recommend the best range of risk and reward for each person individually. These numbers would help provide different zoning with different risk range and or trading style for users you choose, helping to avoid creating any chaos in the market or a particular asset or stock, in case of providing the same price range to a bigger crowd.

The platform includes a bio-feedback module for collecting all the body responses associated with stress, psychology of money, fast decision making under pressure of losing capital and more. We expect these data provide extremely valuable insights from human body in real-time while actively trading and investing in the financial market. Biosensing data such as heartbeat, stress level, blood pressure, and EKG and other data will be collected, used and plotted to monitor its pattern and impact against human decisions. This module also aims to help a user make more robust and consistent decisions while minimizing the risk.

Heartbeat data is the most sensitive biofeedback or natural body response to imminent stress, or risk responding mechanisms especially when it comes to losing and releasing adrenaline, or dopamine out of fear of loss, or excitement of rewards, respectively. We aim to measure, monitor, and control these abnormal behaviors (e.g. fear, and greed) and bring it to users attention in real-time while under a lot of stress to avoid making emotional decisions out of fear or overconfidence which both lead to substantial financial and capital loss.

The collected bio-feedback data including heartbeat, and analyzes the data and plots it against the market price or assets under trading in real-time. It also shows how biofeedback signal (e.g. in this case heartbeat) data is collected, analyzed and plotted. Data can be plotted as a raw as well as the analysis of the biofeedback can be also provided in the form of plot.

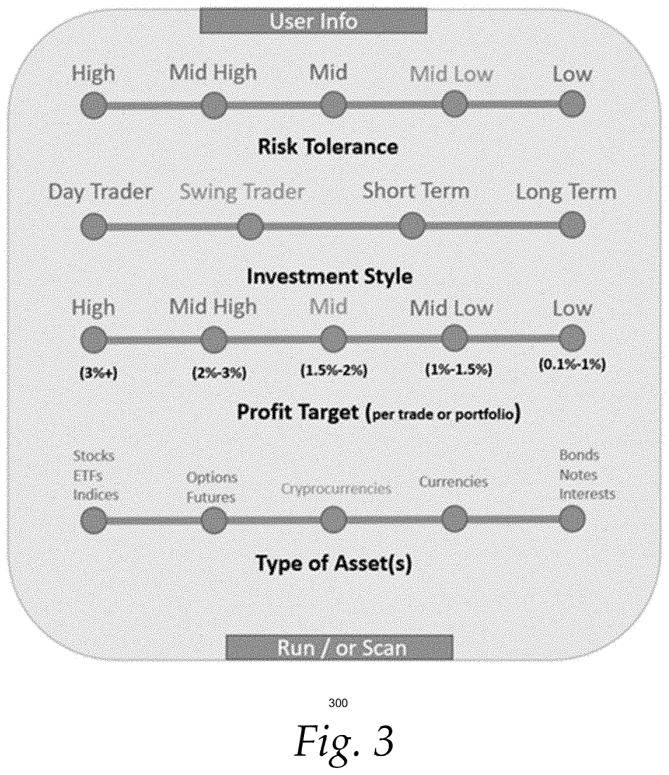

FIG. 3 shows a panel with user preferences. The platform may adapt the trading style of a user to a simple parameterization of the user preferences. As such, user preferences may include investment style, risk tolerance, profit expectation and target, and the type of asset. The user preferences, for example, are used to customize investment and trading opportunities. The user preferences can be applied to certain asset classes or markets, domestically and internationally. For example, user can choose US Stock market, and other financial and capital markets.

The platform provides the daily analysis of the optimum targets depending on the available information and the user data. The platform will give to the user the following information, which is the result of the analysis in the back-end analytical platform. For example, the 10 most interesting assets, the optimum time frame to monitor the stock based on global trend (long or short), the estimated risk and target benefits together with the suggested stops. Based on these pieces of information approved (and/or modified) by the user, the platform will provide to the user the optimum entrance and exit signals.

Learning procedure of the analysis is done in a multi-time frame fashion. The current quantitative measurements of risk and rewards, and therefore statistical index of the Artificial Intelligence algorithm calculates the statistical distribution function based on Value at Risk parameter and combines it with predictive analytics of uptrends and downtrends to come up with the best BUY and SELL opportunities.

FIG. 4 shows an exemplary architecture of a stock selection module. The architecture illustrates how a stock is correlated to 3 stock headers in a typical time, length, and frame. The correlation tree for a given stock may be provided based on Kruskal's algorithm. Other algorithms may also be useful. For example, FIG. 4 shows the relationship between the selected stock 1 with the 3 most correlated headers with both positively and negatively correlations. In this case, two stocks are positively correlated and one stock exhibits a negative correlation.

The platform establishes for the selected asset the mostly correlated assets in the selected time-frame, positively and negatively. For that purpose, the platform uses different measures of dependency in a typical time length that it can be automatically established based on mathematical modeling of time series. This information can be shown as a graph in a dependency tree (minimum spanning tree) by finding the header stocks that exhibit the biggest correlation with the target. The method uses different kinds of correlations (e.g., Pearson, Sperman's rank correlation, Mutual information, etc.) to establish the existing dependencies among stocks. These correlations will serve to establish confirmations about the entrance/exit signals. Based on this analysis, the method provides the matrix of investment opportunities.

FIG. 5 shows an exemplary matrix of investment opportunities for stock selection based on the analysis performed by the platform. The matrix, for example, may be a heatmap based on the ranking of stocks and other assets based on their size as well as measured parameters.

The matrix shows how opportunities may be ranked based on buy, sell and hold, horizontally as well as their risk ranking from least risky to most risk vertically. The table may be sorted out or ranked based on any parameter of choice, such as reward to risk ratio, holding period, risk %, timeframe, etc. The table may be color-coded based on the strength for BUY, SELL, and HOLD as well. For example, below vertical columns for BUY, HOLD, and SELL or SHORT, opportunities are ranked based on Reward to Risk ratio. It may be ranked and sorted based on any other parameter of choice by user, listed in the table. A small graph may be also displayed in each square of the table which is representative of the corresponding symbol. The matrix also shows the optimum time frame to monitor the stock and also to produce the entrance and exit signals dynamically.

Sentiment analysis is employed to confirm these signals. Multiple correlations to different kind of signals will be established and dynamically updated. These signals might come from social networks and/or different economic indicators that can be weakly correlated to the target, which might be sampled at a different rate than the corresponding target. The sentiment analysis signals might be also delayed with respect to the optimum entrance and exit signals provided by the values of the stock, the volume information and the bid and ask. A machine learning methodology will be implemented to take into account and correct this delay.

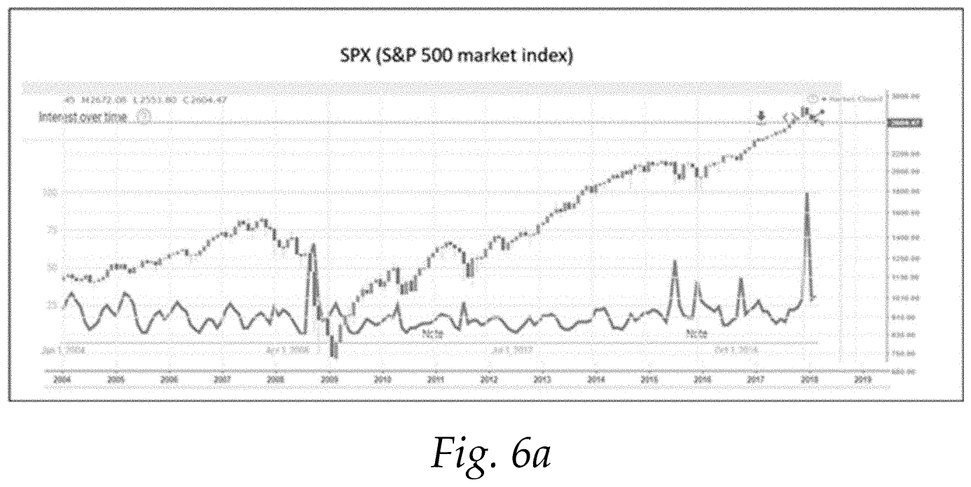

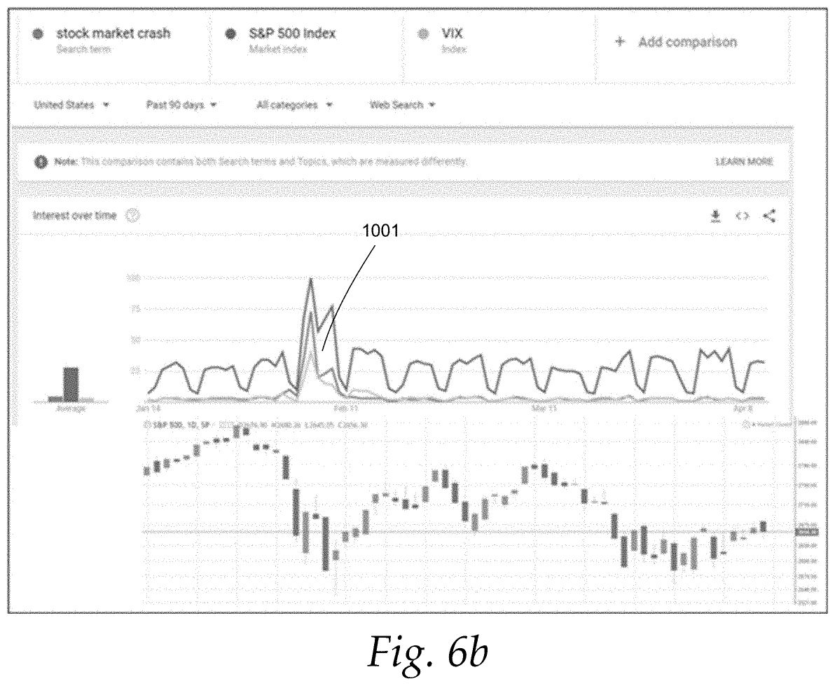

Referring to FIGS. 6a-6b, Sentiment analysis may use different signals from social networks and other economic indicators. Sentiment analysis will be also used to confirm these signals. Multiple correlations to different kind of signals will be established and dynamically updated. These signals might come from social networks and/or different economic indicators that may be weakly correlated to the target, which might be sampled at a different rate than the corresponding target. The sentiment analysis signals might be also delayed with respect to the optimum entrance and exit signals provided by the values of the stock, the volume information and the bid and ask. A machine learning methodology will be implemented to take into account and correct this delay.

FIG. 6a depicts the overlapped S&P 500 monthly price chart on sentiment measurements curve for the following keyword “stock market crash” from 2004 to present. Sentiment measurements in this graph is a normalized number of times that the keyword is searched and/or repeated in financial news, reports, and other alternative sources of data on the web. Sentiment graph based on financial, and fundamental variables of an asset or news from decision making agencies have a big impact on the reaction of users and investors on a particular asset or on the market as a whole. These reports are Federal Reserves, European Central Bank, IMF, etc. reports and updates on economical fundamental changes such as unemployment or inflation, deflation etc. or reports from companies to tax and regulatory agencies bodies such as SEC, FINRA, etc. Also pending litigations, management changes, board decisions, executive team's shares buying or sell outs would affect the price of an asset. Moreover, the sentiment of analytics from different institutions and their reports and analysis on upgrading, or downgrading certain assets such as stocks would also affect the sentiment and behaviour of the investors over certain assets, sectors, or industries.

The platform will also learn using machine learning and optimization techniques which are the optimum attributes that confirm the optimum entrance and exit signals. This analysis includes, but it is not limited to, global optimization algorithms such Pattern Recognition, and Neural Networks Techniques. Particularly we will use Long Short Term Memory (LSTM) neural networks that are commonly used for the prediction of the stock price.

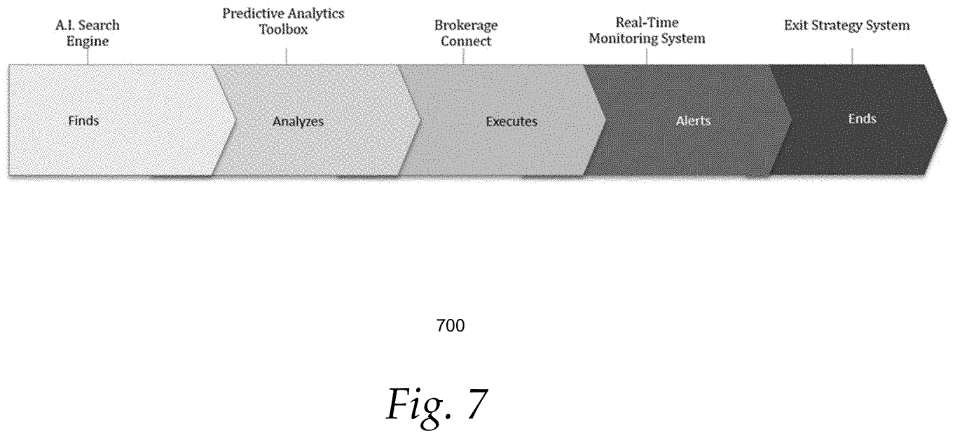

FIG. 7 shows an exemplary embodiment an intelligent trading risk management process. At each step, the risk associated with decision making and analysis and execution are managed and minimized.

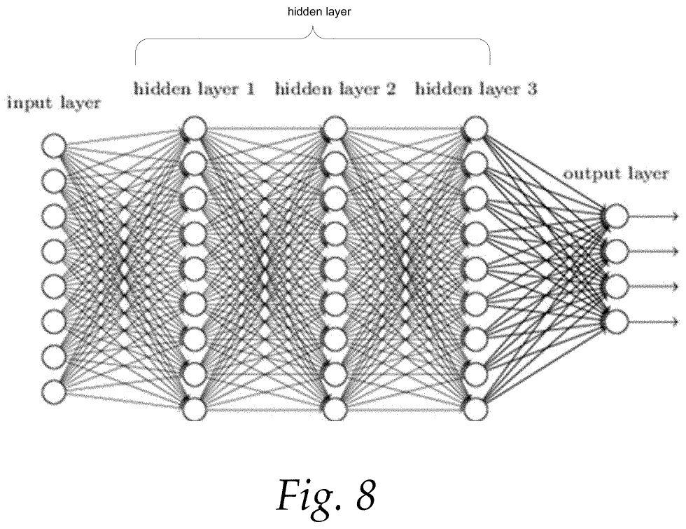

FIG. 8 shows an embodiment of a deep learning architecture for stock prediction. The input layer will be based on price information, different technical descriptors, volume, bid and ask, sentiment analysis, etc. The Neural Network will predict the future value, the trend, and other interesting features that will be the result of the output layer. The architecture will be optimized using Particle Swarm Optimization that will serve to explore the space of equivalent architectures. The final decision will be made by majority voting.

All the decisions are given with its corresponding uncertainty assessment. The platform will give entrance and exit signal for long and short trading taking into account the information and profile given by the user. Sometimes the signals might not be optimum since the user is given very conservatory risk tolerance and benefit target information. Nevertheless, the last decision is always user-based (semi-automatic mode).

The method also has the option of the automatic mode to trade the selected stocks without the user decision. This automatic mode has the advantage of avoiding fear and greed, since it is based in purely quantitative analysis.

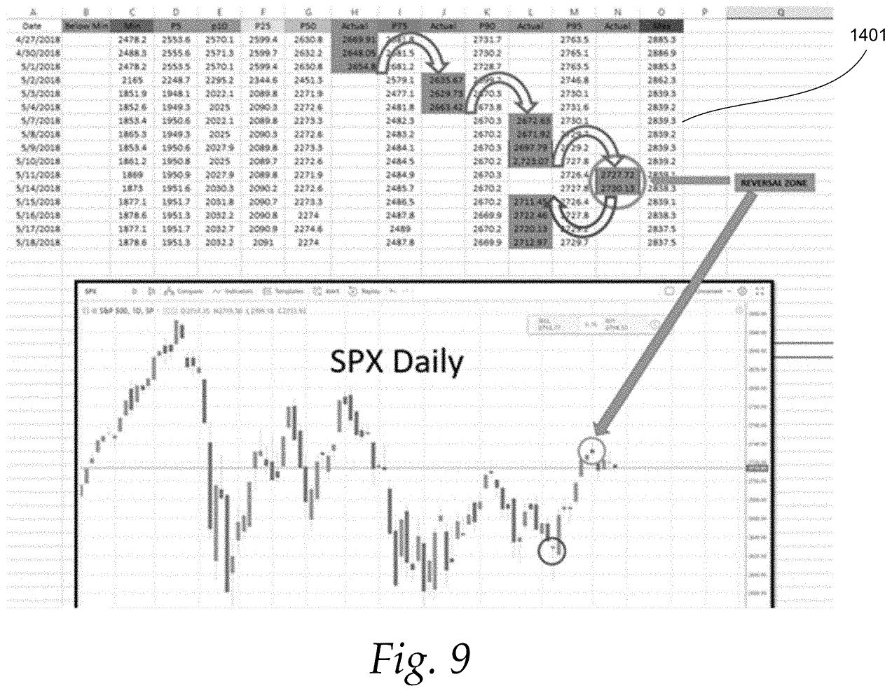

Future predictions may be provided in different time frames (minutes, days or weeks). For example, as shown in FIG. 9, future prediction for 16 weeks for SP500 is illustrated. In this case, the CDFs are shown in a matrix and the new stocks values switch on different cells of the prediction matrix

Different sources of data, such as supply and demand function and behaviors, news, reports and events, would affect every single asset's behavior in different markets. These factors create market volatility and uncertainty at any moment in time. These data and behavior impose uncertainty in investment decision making processes. The framework aims to resolve uncertain sources of information which impose risk on every single trade, portfolio and/or asset executed by the end-users, domestically within the US and/or globally. The comprehensive and easy-to-use quant framework also helps in managing risks for globally allocated portfolios and assets. The framework is able to identify, assess and manage risk in a single trade to big scale assets intelligently and provide real-time analytics for end-users to minimize the risk of entering and exiting a trade, market, and/or an asset.

The intelligent trading and risk management framework is able to provide accurate signaling for entry and exit, during buying and/or selling of low volatility to very high volatility assets. The framework, based on intelligent analytics, also provides a zone in order to buy or sell assets which would help in mitigating execution as well as timing risk. The end-user may or may not miss an opportunity to make a decision at a point in time, but in this zone the end-user is provided time for making a decision to buy or sell, without the feeling of missing out which may typically result in the end-user chasing a price action. The framework enables the end-user to avoid chasing a price action which is usually one of the main factors and reasons for making wrong decisions and mistakes.

Screening of investment and trading opportunities are the essential parts of the method. Combined quantitative models based as well as sentiment measurements are the backbone of finding the best opportunities and risk ranking system of the method. Key modules of the method include a cutting-edge revolutionary trading framework to achieve optimal investment decisions. Disruption at its core, the method helps users in order to maximize the number of right decisions (e.g. profit) with very high accuracy (more than 90%), minimizing the number of wrong decisions (e.g. amount of loss of capital).

The framework provides a Risk Optimal Decision Making methodology for financial instruments based on different modules: 1) AI based risk ranking system in real time; 2) Uncertainty bands and Predictive Analysis; 3) Big data and Sentiment Analysis; and 4) Intelligent Portfolio selection and optimization. The method uses an in-house Optimal Investment Stocks Finder, based on the user information: risk tolerance, investment style, profit target and type of preferred asset. The framework will establish the optimal entrance and exit signals for the selected asset. Further, this framework can work in semi-automatic and automatic mode, without any input from the user.

Measuring heartbeat for many people, and in a bigger scale of population would also give an indicator of how a crowd behavior, risk, fear, greed would react to the market and vice versa. This would be a very powerful measure in terms of how the crowd is affected by market price action and behavior and how market is affected by a big number of traders whom their body signals are measured and analyzed in real-time versus the market.

Wearables of all sorts may be provided for this integrated trading and risk management framework. The wearables are adaptable to be paired to the integrated trading and risk management framework, e.g. via an app, to make the following measurements a reality: Heartbeat in BPM, Blood Pressure, EKG, EEG, EMG, etc.

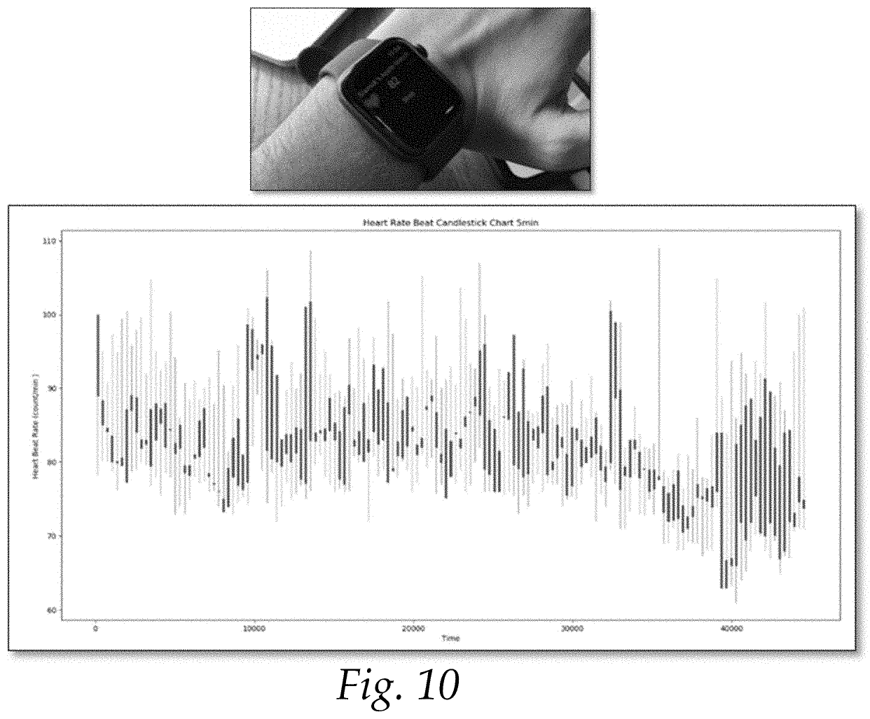

FIG. 10 shows an embodiment of a device for recording information of the user. As shown, the device is recording the heartbeat of a user versus price chart in time and magnitude. Various types of wearable devices are available, such as Apple watch, Fitbit or other types of wearable devices.

Furthermore, a hat may be provided for this integrated trading and risk management framework. AR/VR through hat is going to bring everything in user's fingertips and eyes, through its hat. The hat is able to wirelessly communicate and analyze market data, user data, and any other sort of relevant data types which are going to be used through the process.

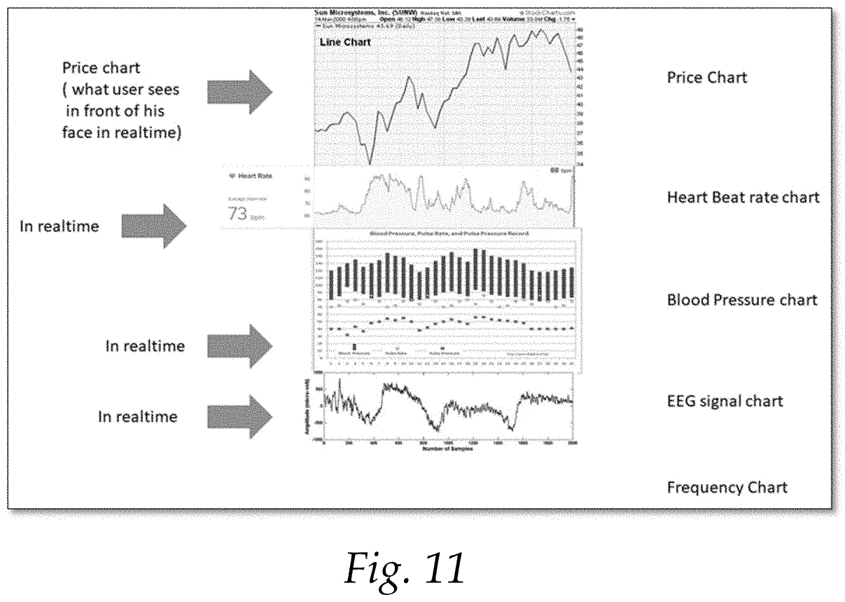

FIG. 11 shows a correlation of heart rate, blood pressure, EEG and price chart in time.

The present disclosure may be embodied in other specific forms without departing from the spirit or essential characteristics thereof. The foregoing embodiments, therefore, are to be considered in all respects illustrative rather than limiting the invention described herein. The scope of the invention is thus indicated by the appended claims, rather than by the foregoing description, and all changes that come within the meaning and range of equivalency of the claims are intended to be embraced therein.

What is claimed is:

1. A method for trading and risk management, the method comprising:
identifying quantitative and sentimental parameters of trading market;
receiving bio-signals including natural responses of a user;
analyzing the quantitative and sentimental parameters of the trading market;
providing a stock selection module to perform future predictions based on the analyzed quantitative and sentimental parameters, wherein the stock selection module comprises a deep learning architecture;
providing a probability number in percentage to a trading decision; and
providing entrance and exit signals to the user based on the user's preferences, and wherein the received bio-signals are configured to verify the trading decision and customize the analyzing based on the user's preferences.
